(19) United States
(12) Patent Application Publication
(10) Pub. No.: US 2025/0073293 A1
(43) Pub. Date: Mar. 6, 2025

(54) Method for preventing hair loss or promoting hair growth using a composition containing an extract of a flower of a Philadelphus schrenckii

(71) Applicant: STOLIVE Co.,Ltd, Gangwon-do (KR)

(72) Inventors: Ji-Hyung CHUNG, Seoul (KR); Jun Kyoung KIM, Seoul (KR)

(21) Appl. No.: 18/814,573

(22) Filed: Aug. 25, 2024

Related U.S. Application Data
(63) Continuation of application No. PCT/KR2023/002368, filed on Feb. 20, 2023.

(30) Foreign Application Priority Data
Feb. 24, 2022 (KR) 10-2022-0024098

(51) Int. Cl. A61K 36/185; A61K 8/9789; A61P 17/14; A61Q 7/00

(52) U.S. Cl. CPC A61K 36/185; A61K 8/9789; A61P 17/14; A61Q 7/00

(57) ABSTRACT

The present invention provides a method for preventing hair loss or promoting hair growth using a flower extract of Philadelphus schrenckii.

### CROSS-REFERENCE TO RELATED APPLICATION

This application is a US Bypass Continuation Application of International Application No. PCT/KR2023/002368, filed Feb. 20, 2023, which claims priority to and the benefit of Korean Patent Application No. 10-2022-0024098, filed Feb. 24, 2022, the disclosure of which is incorporated herein by reference in its entirety.

### BACKGROUND

### Technical Field

The present invention relates to a method for preventing hair loss or promoting hair growth using a flower extract of Philadelphus schrenckii.

### Background Art

Hair follicles, physiological organs in the body that produce hair, repeat the hair growth cycle, which is divided into the anagen phase in which hair is produced and the actively growing hair during the developmental process after birth, the catagen phase in which hair degenerates, the telogen phase until hair falls out, and the exogen phase in which hair loss occurs, and are involved in the growth, maintenance, and loss of hair. The activity of hair follicles occurs by dermal papilla cells, and in particular, the proliferation and differentiation of dermal papilla cells are primarily involved in the progression of the hair growth cycle and hair formation (Botchkarev V A, Kishimoto J, Molecular control of epithelial-mesenchymal interactions during hair follicle cycling. J Investig Dermatol Symp Proc 8, 46-55, 2003). During the growth phase when hair actively grows, active proliferation and differentiation of hair papilla cells occur, and during the catagen, telogen, and alopecia phases when hair growth stops and hair loss occurs, these cells die (Botchkarev V A, Batchkareva N V, Nakamura M, Noggin is required for induction of the hair follicle growth phase in postnatal skin. FASEB J 15, 2205-2214, 2001). In addition, cell division and migration near the hair papilla are closely related to hair growth, and new hair is generated from the hair papilla during the growth phase. Various cytokines, hormones, etc. activate cells, causing cell migration into the hair papilla, which affects hair growth (Eva M J Peters, Desmond J Tobin, Migration of melanoblasts into developing murine hair follicle is accompanied by transient c-kit expression, J Histochem Cytochem, 50, 6751-766, 2002; Jahoda C A, Cell movement in the hair follicle dermis-more than a two-way street J Invest Dermatol, 1221, 1523-1747, 2003). Therefore, since the proliferation and death of hair papilla cells are closely related to hair growth and hair loss, inducing their proliferation to prolong the growth phase or inhibiting cell death to shorten the regression phase, resting phase, and hair loss phase will be a way to improve and treat hair loss.

Currently, representative drugs known to have hair loss suppression and hair growth promotion effects include minoxidil, finasteride, biotin, and dutasteride, and they are commercially available in various forms such as pharmaceuticals, shampoos/tonic products, and medical devices. In addition to synthetic compounds, the use of proteins/peptides, stem cells, and their culture solutions has been suggested for hair loss suppression and hair growth promotion. In addition, the use of various natural extracts with excellent safety has also been suggested.

### SUMMARY

The present inventors searched for various extracts derived from natural products that have hair loss prevention or hair growth promotion activities. As a result, it was found that an extract obtained from the flower of Philadelphus schrenckii significantly increases the proliferation of hair papilla cells, which play an important regulatory role in the development of hair follicles and hair growth and cycle control, the expression of growth factors and cytokines related to the hair growth phase, and the activation of Wnt/β-catenin signaling proteins.

Accordingly, the present invention aims to provide a method for preventing hair loss or promoting hair growth using a flower extract of Philadelphus schrenckii.

According to one aspect of the present invention, a method for preventing hair loss or promoting hair growth using a flower extract of Philadelphus schrenckii is provided.

According to one aspect of the present invention, a composition for external application to the skin for preventing hair loss or promoting hair growth, comprising a flower extract of Philadelphus schrenckii as an effective ingredient, is provided.

According to another aspect of the present invention, a cosmetic composition for preventing hair loss or promoting hair growth is provided, comprising a flower extract of Philadelphus schrenckii as an active ingredient.

According to one aspect of the present invention, a weak composition for preventing hair loss or promoting hair growth is provided, comprising a flower extract of Philadelphus schrenckii as an active ingredient.

According to one aspect of the present invention, a food composition for preventing hair loss or promoting hair growth is provided, which comprises a flower extract of Philadelphus schrenckii as an effective ingredient.

According to the present invention, it was revealed that the flower extract of Philadelphus schrenckii increases the proliferation of hair papilla cells, increases cell size in three-dimensional culture of hair papilla cells, and significantly increases the expression of growth factors and cytokines related to the hair growth phase and the activation of Wnt/β-catenin signaling proteins. Therefore, the flower extract of Philadelphus schrenckii can be usefully used in a composition for preventing hair loss or promoting hair growth.

### BRIEF DESCRIPTION OF THE DRAWINGS

FIG. 1 shows the results of evaluating the proliferation efficacy of human dermal papilla cells according to treatment with various concentrations of flower extract of Philadelphus schrenckii.

FIG. 2A shows the results of photographing changes in cell size due to treatment with flower extract of Philadelphus schrenckii in a three-dimensional culture of human breast papilla cells.

FIG. 2B shows the results of measuring the change in cell size by treatment with a flower extract of Philadelphus schrenckii in a three-dimensional culture of human breast papilla cells.

FIG. 3A shows the results of evaluating an expression of growth factors and an expression of cytokines related to the hair growth period in human hair papilla cells by immunoblotting analysis method after treatment with various concentrations of flower extract of Philadelphus schrenckii.

FIG. 3B shows the results of evaluating Wnt/β-catenin signaling protein activation in human breast papilla cells by immunoblotting analysis method after treatment with various concentrations of flower extract of Philadelphus schrenckii.

FIG. 4A shows the results of evaluating an expression of VEGF, and an expression of a growth factor related to the hair growth period, in human hair papilla cells by immunoblotting analysis method after treatment with various concentrations of flower extract of Philadelphus schrenckii.

FIG. 4B shows the results of evaluating the expression of IGF-1, a growth factor related to the hair growth period by immunoblotting analysis method, after treatment with various concentrations of the flower extract of Philadelphus schrenckii in human hair papilla cells.

FIG. 5 shows the results of evaluating IL-6 expression in human breast papilla cells by immunoblotting analysis method after treatment with various concentrations of flower extract of Philadelphus schrenckii.

FIG. 6 shows the results of evaluating the expression of BMP2, a hair growth inhibitory factor by immunoblotting analysis method after treating human hair papilla cells with various concentrations of flower extract of Philadelphus schrenckii.

### DETAILED DESCRIPTION

In the specification of the present disclosure, “hair loss” includes not only hair falling out from the scalp, but also a decrease in hair density or hair thickness.

In the specification of the present disclosure, “stimulating hair growth” includes stimulating the creation of new hair and maintaining and growing existing hair in a healthy manner.

In the specification of the present disclosure, “prevention” means any act of inhibiting or delaying the occurrence or onset of one or more symptoms associated with hair loss by administering the composition of the present invention, and does not necessarily mean complete inhibition of the onset of hair loss.

In the specification of the present disclosure, “treatment” means any action by which one or more symptoms associated with hair loss are suppressed, reduced or improved in a subject who is or was previously exhibiting symptoms, by administering a composition of the present invention.

In the specification of the present disclosure, “administration” means introducing a composition of the present invention into a subject by any method. The route of administration includes oral or parenteral administration by any route that can reach the target tissue. For example, parenteral administration, such as topical application by application, can be applied, and the composition of the present invention can also be administered by any device that allows it to travel to target cells.

According to embodiments of the present disclosure, a method for promoting hair growth or preventing hair loss may include administering to a subject a composition comprising an effective amount of a Philadelphus schenckii extract.

Here, the method can exhibit effects of promoting hair growth, preventing hair growth, or treating hair loss in a subject.

Here, the subject may include a subject in need of hair growth promotion or hair loss prevention, and the subject may be an animal, and may include a primate including a human.

According to embodiments of the present disclosure, the composition may proliferate hair follicle cells of a subject. The hair follicle cells may be dermal papilla cells.

According to embodiments of the present disclosure, the composition is Wnt/β-catenin of a subject The activity of signal transduction cascades can be modulated by activating signal transduction proteins. According to embodiments of the present disclosure, the composition may increase the expression of at least one selected from the group consisting of vascular endothelial growth factor (VEGF), Versican, transforming growth factor beta 2 (TGFβ2), hepatocyte growth factor (HGF), glycogen synthase kinase-3 beta (GSK3β), β-catenin, and Wnt5α.

Here, VEGF (Vascular endothelial growth factor) is a vascular endothelial growth factor that improves blood circulation by growing the vascular endothelium, thereby enhancing hair growth and differentiation of hair root cells. IGF-1 (Insulin-like growth factor) is known to promote hair growth by promoting the growth of hair follicles and epithelial cells and preventing apoptosis of hair cells. Human dermal papilla cells are fibroblasts derived from the mesoderm, and are reported to affect the hair cycle by secreting proteins such as insulin-like growth factor-1 (IGF-1), hepatocyte growth factor (HGF), and fibroblast growth factor-7 (FGF-7).

The activity of the Wnt/β-catenin signaling pathway is known to play a role in skin formation and hair follicle development, and hair growth is known to be inhibited in mice in which the Wnt gene is knocked down. Therefore, a substance that promotes the activation of the Wnt/β-catenin signaling pathway present in hair follicle stem cells can promote hair growth.

The present invention provides a composition for preventing hair loss or promoting hair growth, comprising a flower extract of Philadelphus schrenckii as an active ingredient.

According to embodiments of the present disclosure, the flower extract of Philadelphus schrenckii may increase the proliferation of hair papilla cells, increases cell size in three-dimensional culture of hair papilla cells, and significantly increases the expression of growth factors and cytokines related to hair growth phase and the activation of Wnt/β-catenin signaling proteins. Therefore, the flower extract of Philadelphus schrenckii may be usefully used in a composition for preventing hair loss or promoting hair growth.

Philadelphus schrenckii is distributed in Korea, Japan, or China, and mainly grows in mountain valleys. It is 2-4 m tall, and the leaves on the small branches are opposite, ovate or elliptic, pointed at both ends, and have indistinct serrations. In April to May, white flowers bloom in racemes in the leaf axils or tops, with fine hairs, and 5 to 7 flowers bloom. The calyx has fine hairs at the inner end, and the petals are round. There are four styles, and the fruit is a round capsule that ripens in September. The wood is used for ornamental purposes, and the young leaves are edible. The unripe fruit and roots are known to be effective for hemorrhoids, and the flowers are known to be effective as a nervous system tonic or diuretic.

Here, the Philadelphus schrenckii flower extract may include all substances obtained by extracting components of the Philadelphus schrenckii flower, regardless of the extraction method, extraction solvent, extracted component, or form of the extract. The Philadelphus schrenckii flower extract may include substances obtained through an extraction method that include a process of treating with heat, acid, base, enzyme, etc. in the process of extracting the components. In addition, the Philadelphus schrenckii flower extract may include all substances that can be obtained by processing or treating the substances obtained by extracting components of the Philadelphus schrenckii flower by another method after extraction.

According to embodiments of the present disclosure, the invention can be obtained by performing an extraction process including a step of extracting a powdered product of a deciduous tree flower, for example, a powdered product obtained by drying and crushing deciduous tree flowers, with an extraction solvent.

The above powdered product of the Ginkgo biloba flower can be obtained by drying and crushing the Philadelphus schrenckii flower in a manner and turning it into a powder. In addition, the extraction solvent can be used in a ratio of 15 to 100 mL per 1 g of the powdered product of the Philadelphus schrenckii flower, but is not limited thereto.

The extraction solvent of the Philadelphus schrenckii flower extract includes water, an organic solvent, or a mixed solvent of water and an organic solvent. The water includes distilled water and purified water, and the organic solvent may include at least one selected from the group consisting of a C₁ to C₆ lower alcohol, acetone, ether, ethyl acetate, diethyl ether, ethyl methyl ketone, and chloroform. For example, the solvent includes water, a C₁ to C₄ Alcohol, and a mixed solvent of water and a C₁ to C₄ alcohol.

In one example, the extraction process is not particularly limited in number or method of extraction or fractionation, for example, cold extraction, ultrasonic extraction, reflux cooling extraction, hot water extraction. Extraction methods such as hot water extraction may be used.

The extraction process may be performed, for example, by performing an extraction treatment at a temperature of 60 to 100° C. and evaporating the extraction solvent. The extraction process may be performed once or multiple times (for example, twice). For example, the extraction process may be performed by adding an extraction solvent to the ground material of the flowers of the Philadelphus schrenckii, performing an extraction treatment at a temperature of 60 to 100° C. and evaporating the extraction solvent to obtain a first extract, further adding an extraction solvent, performing an extraction treatment at a temperature of 60 to 100° C. and evaporating the extraction solvent to obtain a second extract, and then concentrating the first and second extracts to an preset concentration. Additionally, impurities may be removed by filtering through a filter paper (for example, a 0.45 μm filter paper). The extract may preferably be stored under a refrigerator. In addition, the extract may be manufactured in the form of oil (i.e., oily form) or liquid, powder, or solid by drying according to a method such as reduced pressure drying, hot air drying, or freeze drying.

In one embodiment of the present disclosure, the composition may be a composition for external use in the skin.

In one example, the skin external application composition may be a cosmetic composition.

The cosmetic composition may be formulated containing a cosmetically or dermatologically acceptable medium or base. The cosmetic composition may be any formulation suitable for topical application, for example, in the form of a solution, a gel, a solid, a pasty anhydrous product, an emulsion obtained by dispersing an oil phase in an aqueous phase, a suspension, a microemulsion, a microcapsule, a microgranule, or an ionic (liposome) and nonionic vesicular dispersion, or in the form of a cream, a skin, a lotion, a powder, an ointment, a spray, or a concealer stick. The cosmetic composition may also be used in the form of a foam or in the form of an aerosol composition further containing a compressed propellant. These compositions may be manufactured according to conventional methods in the art. In addition, the composition according to one embodiment of the present disclosure may contain a skin absorption promoting substance in order to increase the skin improvement effect. The content of the flower extract of the above-mentioned Philadelphus schrenckii may be contained in an amount effective to achieve the effect of preventing hair loss or promoting hair growth, for example, in an amount of 0.001 to 10 wt % based on the total weight of the composition.

In one example, the cosmetic composition can be used as a composition for the scalp or hair, etc. Accordingly, the composition can be formulated as a formulation for use together with the scalp and hair, for example, a shampoo, rinse, scalp treatment, hair treatment, combined scalp and hair treatment, hair treatment for preventing hair loss, treatment for damaged hair, leave-in conditioner, wash-off conditioner, or hair essence, or a scalp or hair, scalp and hair combined product, etc.

The cosmetic composition may contain various ingredients that are incorporated into a scalp or hair composition according to the various formulations described above or according to a final purpose, and the types and amounts of these ingredients can be easily selected by those skilled in the art.

In addition, the composition may contain adjuvants commonly used in the field of cosmetology or dermatology, such as fatty substances, organic solvents, solubilizers, thickeners, gelling agents, emollients, antioxidants, suspending agents, stabilizers, foaming agents, fragrances, surfactants, water, ionic or nonionic emulsifiers, fillers, sequestering agents, chelating agents, preservatives, vitamins, blocking agents, humectants, essential oils, dyes, pigments, hydrophilic or lipophilic active agents, lipid vesicles or any other ingredients commonly used in cosmetics. The adjuvants may be included in an amount commonly used in the field of cosmetology or dermatology, for example, from 0.001 wt. % to 5 wt. %, for example, from 0.001 wt. % to 3 wt. %, based on the total weight of the composition.

In one embodiment of the present disclosure, the composition may be a pharmaceutical composition.

The pharmaceutical composition may include a pharmaceutically acceptable carrier, and may be formulated as a parenteral dosage form such as a solution, suspension, emulsion, lotion, ointment, lyophilizer, etc. according to a conventional method. Preferably, the pharmaceutically acceptable carrier may be formulated as a transdermal dosage form such as an external solution, emulsion, ointment, etc. The pharmaceutically acceptable carrier includes an aqueous diluent or solvent such as phosphate buffered saline, purified water, sterilized water, and a non-aqueous diluent or solvent such as propylene glycol, polyethylene glycol, and olive oil. In addition, the composition may include a wetting agent, a fragrance, a preservative, etc., as necessary. The dosage of the flower extract of Philadelphus schrenckii contained in the pharmaceutical composition may be appropriately selected by a person skilled in the art depending on the condition of the patient, the degree of the disease, the route and period of administration. The flower extract of Philadelphus schrenckii can be administered (e.g., applied) to the scalp, for example, in an amount of 0.1 to 100 mg per day, and the administration can be administered once a day or in several divided doses.

In one embodiment of the present disclosure, the composition may be a food composition. The composition may be provided as various forms of food additives or functional foods containing the extract of the flower of the Philadelphus schrenckii. The composition may be processed into fermented milk, cheese, yogurt, juice, probiotics, and health foods containing the effective ingredient, and may be used in the form of various other food additives.

In one embodiment, the composition may be a health food composition. The health food composition according to an embodiment of the present invention may contain 0.0001 wt % to 99 wt %, for example, 0.01 wt % to 60 wt %, of a flower extract of Philadelphus schrenckii based on the total weight of the composition, but is not limited thereto. In an embodiment, the health food composition may be formulated into a pill, a capsule, a tablet, a granule, a caramel, or a drink. In another embodiment, the composition may be processed into a form of a liquid, a powder, a granule, a tablet, or a tea bag. The composition may be administered in various ways, such as simple drinking, injection, spraying, or squeezing.

The composition may contain other components, etc., which may have a synergistic effect on the main effect, within a range that does not impair the main effect of the present invention. For example, additives such as fragrances, pigments, bactericides, antioxidants, preservatives, moisturizers, thickeners, inorganic salts, emulsifiers, and synthetic polymer materials may be further included to improve physical properties. In addition, auxiliary components such as water-soluble vitamins, oil-soluble vitamins, high-molecular peptides, high-molecular polysaccharides, and seaweed extracts may be further included. The components may be suitably selected and mixed by person skilled in the art according to the formulation or intended use, and the amount of the component may be selected within a range that does not impair the purpose and effect of the present invention. For example, the amount of the components added may be 0.01 wt % to 5 wt %, for example, 0.01 wt % to 3 wt %, based on the total weight of the composition, but is not limited thereto.

An example of a formulation of a composition according to one aspect of the present specification is described below, but it can also be applied to various other formulations, which are not intended to limit the present disclosure but are merely intended to be specifically described.

### [Formulation Example 1] Tablet

100 mg of extract of Example 1 of the present disclosure, 400 mg of lactose, corn 400 mg of starch and Stearic acid 2 mg of magnesium are Mixed. Tablets were manufactured by pressing them into tablets according to the manufacturing method of conventional tablets.

### [Formulation Example 2] Granules

50 mg of extract of Example 1 of the present disclosure, anhydrous crystal 250 mg of glucose and 550 mg of starch are Mixed and are formed into granules using a fluidized bed granulator, and then filled into a bag.

### [Formulation Example 3] Capsule

100 mg of extract of Example 1 of the present disclosure, 400 mg of lactose, corn 400 mg of starch and Stearic acid 2 mg of magnesium are Mixed. Capsules are manufactured by filling gelatin capsules according to the conventional capsule manufacturing method.

[Formulation example 4] Drink

50 mg of the extract of Example 1 of the present disclosure, 10 g of glucose, 0.6 g of citric acid, and Liquid 25 g of oligosaccharide are Mixed, added 300 ml of purified water and filled each bottle with 200 ml. Then the drink was manufactured by sterilizing it at 130° C. for 4 to 5 seconds.

### [Formulation Example 5] Flexible Toner

A flexible toner was manufactured using a conventional method according to the composition described in the table below.

TABLE 1

| Mixing ingredients | Content (weight %) |
|---|---|
| Philadelphus schrenckii Flower Extract | 0.2 |
| glycerin | 3.0 |
| Butylene glycol | 2.0 |
| Propylene glycol | 2.0 |
| Carboxyvinyl polymer | 0.1 |
| PEG-12 Nonylphenyl Ether | 0.2 |
| Polysorbate 80 | 0.4 |
| Ethanol | 10.0 |
| Triethanolamine | 0.1 |
| Preservatives, Colorants, Fragrances | Appropriate amount |
| Purified water | Remaining amount |

### [Formulation Example 6] Nutritious Toner

A nutritious toner was manufactured using a conventional method according to the composition described in the table below.

TABLE 2

| Mixing ingredients | Content (weight %) |
|---|---|
| Philadelphus schrenckiiFlower Extract | 1.0 |
| glycerin | 3.0 |
| Butylene glycol | 3.0 |
| Propylene glycol | 3.0 |
| Carboxyvinyl polymer | 0.1 |
| beeswax | 4.0 |
| Polysorbate 60 | 1.5 |
| Caprylic/Capric Triglyceride | 5.0 |
| Squalane | 5.0 |
| Sorbitasesquioleate | 1.5 |
| Liquid paraffin | 0.5 |
| Cetearyl alcohol | 1.0 |
| Triethanolamine | 0.2 |
| Preservatives, Colorants, Fragrances | Appropriate amount |
| Purified water | Remaining amount |

### [Formulation Example 7] Massage Cream

A massage cream was manufactured by a conventional method according to the composition described in the table below.

TABLE 3

| Mixing ingredients | Content (weight %) |
|---|---|
| Philadelphus schrenckiiFlower Extract | 2.0 |
| glycerin | 8.0 |
| Butylene glycol | 4.0 |
| Liquid paraffin | 45.0 |
| Beta Glucan | 7.0 |
| Carbomer | 0.1 |
| Caprylic/Capric Triglyceride | 3.0 |
| beeswax | 4.0 |
| Cetearyl Glucoside | 1.5 |
| Sesquioleic acid sorbitan | 0.9 |
| Vaseline | 3.0 |
| paraffin | 1.5 |
| Preservatives, Colorants, Fragrances | Appropriate amount |
| Purified water | Remaining amount |

### [Formulation Example 8] Manufacturing of Hair Lotion

Hair lotion is manufactured according to the usual method using the composition listed in the table below.

TABLE 4

| ingredient | Content (weight %) |
|---|---|
| Philadelphus schrenckiiFlower Extract | 2.0 |
| Purified water | Remaining amount |
| glycerin | 3.0 |
| Butylene glycol | 3.0 |
| Liquid paraffin | 7.0 |
| Beta Glucan | 7.0 |
| Carbomer | 0.1 |
| Edelweiss extract | 2.0 |
| Caprylic Capric Triglyceride | 3.0 |
| Squalene | 5.0 |
| Cetearyl glucoside | 1.5 |
| Sorbitan stearate | 0.4 |
| Polysorbate | 1.2 |
| antiseptic | Appropriate amount |
| incense | Appropriate amount |
| pigment | Appropriate amount |
| Triethanolamine | 0.1 |

### [Formulation Example 9] Manufacturing of Shampoo Composition

A shampoo composition was manufactured according to a conventional method using the composition described in the table below.

TABLE 5

| Ingredients (weight %) | Content (weight %) |
|---|---|
| Polyquaternium-10 | 0.5 |
| Disodium laureth | 20 |
| sulfosuccinate | |
| Philadelphus schrenckii | 0.3 |
| Flower Extract | |
| incense | 1.0 |
| Ethylhexylglycerin | 0.1 |
| Cetyl alcohol | 0.5 |
| Sodium chloride | 0.8 |
| Purified water | To 100 |

### [Formulation Example 10] Manufacturing of Hair Treatment

A hair treatment was manufactured according to the conventional method using the composition described in the table below.

TABLE 6

| | Content |
|---|---|
| Classification (weight %) | (weight %) |
| Philadelphus schrenckii | 0.5 |
| Flower Extract | |
| Stearamidopropyldimethylamine | 2 |
| glycerin | 0.5 |
| spices | 0.5 |
| Caprylyl Glycol | 0.03 |
| Cetostearyl alcohol | 5 |
| Lactic acid | 1 |
| Distilled water | to 100 |

Hereinafter, the present invention may be described in detail through examples. However, these disclosures are intended to illustrate the present invention, and the scope of the present invention is not limited to these examples.

### [Example 1] Manufacturing of Extract of the Flower of the Common Maple

Philadelphus schenckii were separated, dried, and ground using a mortar and pestle. To the ground Philadelphus flowers (3 g), 100 ml of purified water was added, and the mixture was evaporated while not boiling (at approximately 80° C.) in a heating stirrer (Corning PC-420) until a total volume was approximately 30 ml. The mixture may be centrifuged at 3,000 rpm for 20 minutes, to obtain 30 ml of a first extract. A mixture of sediment and 50 ml of purified water was heated in the above method and evaporated until a total volume became 20 ml. The mixture was centrifuged at 3000 rpm for 20 minutes to obtain 20 ml of a second extract. The first extract (30 ml) and the second extract (20 ml) were mixed, and concentrated while heating in the same method until a total volume was approximately 3 ml. A stock extract solution was prepared by adding distilled water to the obtained concentrate (i.e., the extract of the flowers of the Philadelphus schrenckii) to adjust the concentration to 1 mg/mL, filtering it through a filter paper, for example 0.45 μm, and then storing it in the refrigerator.

### [Experimental Example 1] Culturing Human Dermal Papilla Cells

Human mammary papilla cells were purchased from Cell Applications, Inc. (USA). The purchased cells were morphologically confirmed by microscopic observation and cultured in a 37° C., 5% CO 2 incubator using DMEM medium (Dulbecco's Modified Eagle's medium) (Hyclone, USA) containing 10% fetal bovine serum (FBS) and 1% antibiotics (penicillin/streptomycin, amphotericin). After 24 hours of cell culture, the existing medium was removed, washed with phosphate buffered saline (PBS), and additional culture was performed by adding the same new medium.

### [Experimental Example 2] Evaluation of Cell Proliferation Efficacy of the Extract of the Flower of the Philadelphus schrenckii on Maternal Papilla Cells

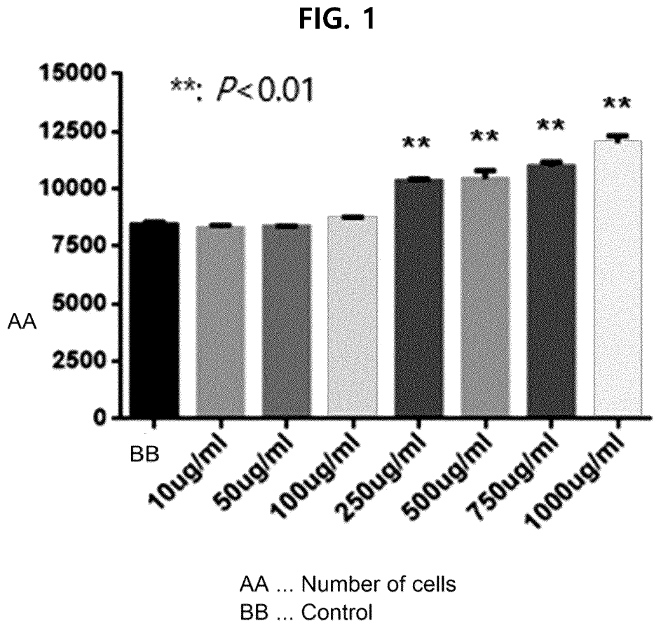

In each well of a 48-well plate coated with collagen exclusively for cell culture, the mammary papilla cells cultured in the Test Example 1 were seeded at 5×10 3 cells/well, and the same medium was added and cultured for 24 hours. The cells were observed under a microscope, the existing medium was removed, and the cells were washed with PBS. Thereafter, new DMEM medium containing 1% FBS and antibiotics was added, and the cells were cultured for an additional 24 hours, and the medium was removed again and washed with PBS. When the new culture medium was added, the Philadelphus schrenckii flower extract prepared in Example 1 was treated to 200 μl of the medium per well so that the final extract concentrations were 10, 50, 100, 250, 500, 750, and 1000 μg/ml, respectively, and then cultured in a 37° C., 5% CO₂ incubator for 24 hours. The control group was treated with the same volume of distilled water. Each experimental group was simultaneously performed in three wells under the same conditions. The cell proliferation efficacy was evaluated by measuring the absorbance at a wavelength of 450 nm using the WST-1 assay (Ez-cytox Cell Viability Assay Kit, Daeilbio, Korea) 24 hours after treatment with the Philadelphus schrenckii flower extract. The results are shown in FIG. 1. As can be seen in the results of FIG. 1, it was confirmed that cell proliferation significantly increased in a concentration-dependent manner at a treatment concentration of 100 μg/ml or higher of the Philadelphus schrenckii flower extract.

### [Experimental Example 3] Three-Dimensional Culture of Maternal Papilla Cells and Evaluation of Cell Size Using Extracts of the Flower of the Philadelphus schrenckii

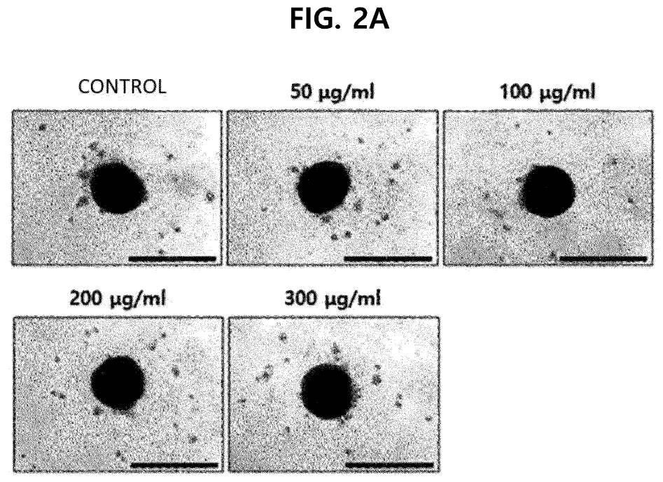

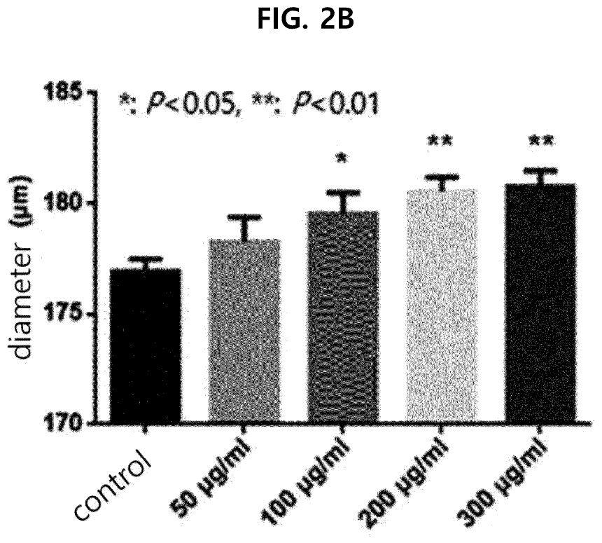

In Experimental Example 1, the cultured mammary papilla cells were seeded at 1.5×10^{4/well} in a 96-well round bottoms with lid ultra-low attachment microplate (Corning, USA) and treated with DMEM medium (Dulbecco's Modified Eagle's medium) (Hyclone, USA) containing 10% fetal bovine serum (FBS) and 1% antibiotics (penicillin/streptomycin, amphotericin) and the extract of the flower of the common hollyhock was added to each well to give a final concentration of 50, 100, 200, and 300 μg/ml per 100 μl of the medium. The cells were clumped together in a shaker for 20 minutes and then cultured for 72 hours. The control group was treated with the same volume of distilled water. Each experimental group was simultaneously performed on 20 wells under the same conditions, and at the end of the culture (72 hours), the cell morphology was photographed using an optical stereomicroscope (SMZ745T, Nikon, Japan) (See FIG. 2A), and the cell diameter was measured respectively and the average was calculated (See FIG. 2B). As shown from the results of FIG. 2A and FIG. 2B, the cell diameter tended to increase in a concentration-dependent manner depending on the treatment concentration of the extract of the flower of the Philadelphus schrenckii.

### [Experimental Example 4] Evaluation of Hair Growth Factor, Cytokine Expression, and Wnt/β-Catenin Signaling Activation in Hair Papilla Cells

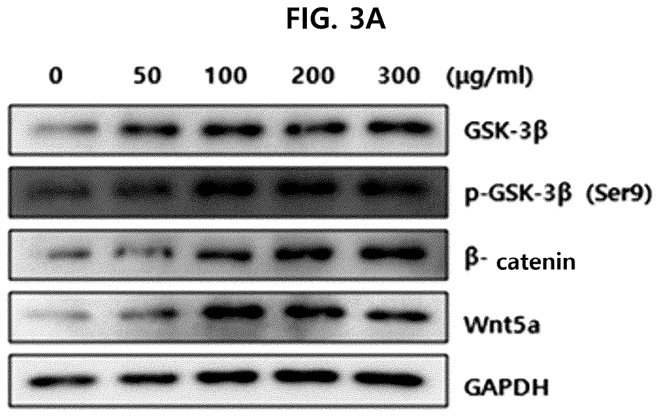

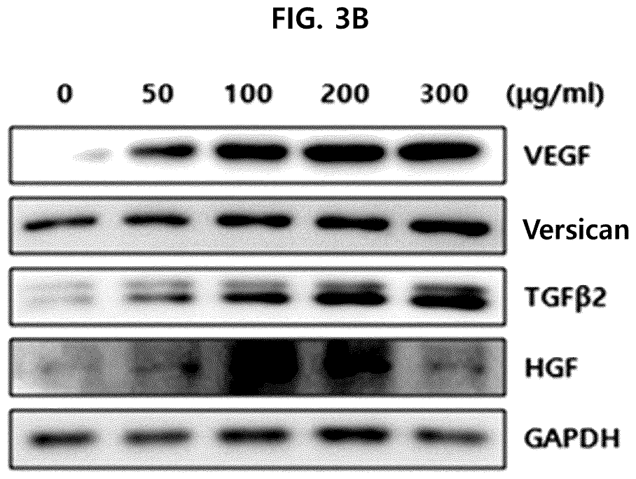

To investigate the efficacy of the extract of the flower of the common dandelion on growth factors, including VEGF and IGF-1, which are representative growth factors known to affect the promotion of regeneration and growth of dermal papilla cells, as well as cytokines and Wnt/β-catenin signaling proteins, an immunoblot analysis method was performed. Dermal papilla cells were seeded at 3×10⁵ cells/well in each well of a 6-well plate containing DMEM medium and cultured in an incubator at 37° C. and 5% CO₂. After 24 hours, the extract of the flower of the Philadelphus schrenckii was treated to a final concentration of 50, 100, 200, and 300 μg/ml per 100 μl of medium in each well, and the medium was removed and washed with PBS. A control group was treated with the same volume of distilled water. Cells were lysed with RIPA buffer (Sigma-Aldrich, USA) containing protease inhibitor cocktail (Sigma-Aldrich, USA) and phosphatase inhibitor cocktail, and centrifuged. SDS-PAGE was performed on the same amount of proteins obtained from the supernatant, and immunoblot analysis was performed using anti-VEGF antibody (Abcam, USA), anti-IGF1 antibody (Abcam, USA), anti-Versican antibody (Abcam, USA), anti-TGFβ2 antibody (Abcam, USA), anti-HGF antibody (Abcam, USA), anti-GSK3β antibody (Cell Signaling Technology, USA), anti-phospho-GSK3β antibody (Cell Signaling Technology, USA), anti-β-catenin antibody (Cell Signaling Technology, USA), and anti-Wnt5α antibody (Abcam, USA). Compared to the control group, the expression levels of cell growth factors and cytokines significantly increased in the group treated with the extract of the flower of the Philadelphus schrenckii (see FIG. 3A), and the protein levels of Wnt5α, GSK3β, and β-catenin, which are representative factors mediating Wnt/β-catenin signaling, increased in the group treated with the extract of the flower of the Philadelphus schrenckii (see FIG. 3B).

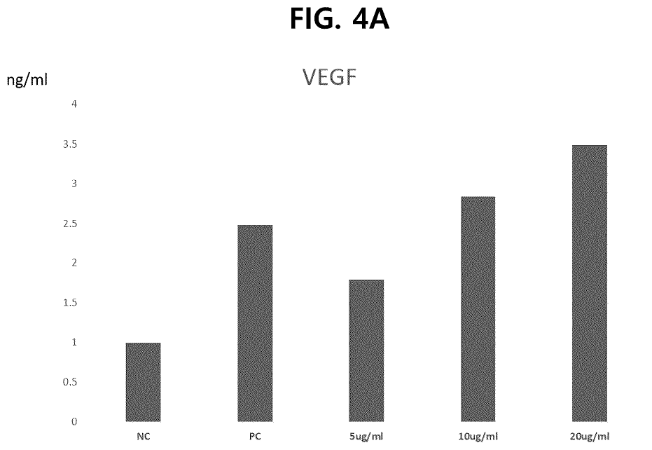

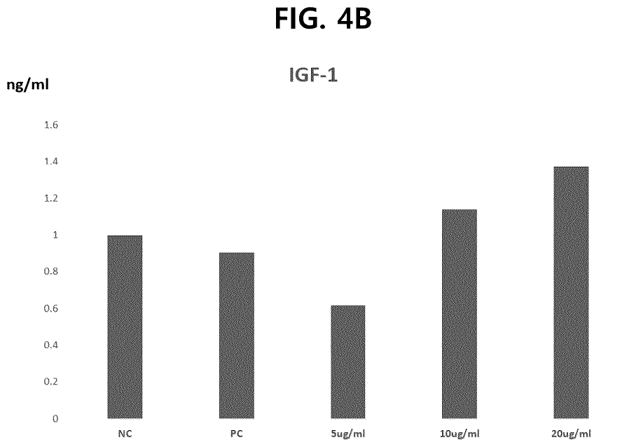

In particular, in the groups treated with the extract of the flower of the Philadelphus schrenckii (5, 10, 20 μg/ml), the expression levels of VEGF (see FIG. 4A) and IGF-1 (see FIG. 4B) increased in proportion to the concentration of the extract of the flower of the Philadelphus schrenckii.

### [Example 2] Manufacturing of Extract of Philadelphus schrenckii Flower

5 g of dried raw Philadelphus schrenckii flowers were added to 100 ml of purified water, stirred at 95° C. for 4 hours, and cooled to 27° C. after hot water extraction. Centrifuged at 4° C., 3500 rpm for 30 minutes, the supernatant was collected, and filtered through a 0.45 μm syringe filter. Purified water was added to make 60 ml of the filtered product, and 35 ml of butylene glycol was added to use it as a Philadelphus schrenckii flower extract sample.

### [Experimental Example 5] Anti-Inflammatory Effect (IL-6, IL-1β)

In each well of a 6-well plate containing DMEM medium, 5×10 4 cells/well of maternal papilla cells (see Test Example 1) were seeded and cultured in a 37° C. in a 5% CO₂ incubator. After culturing for 24 hours, inflammation was induced by pretreatment with serum-free medium and 10 μg/ml PG-LPS (InvivoGen, tlrl-pglps). 16 hours after inflammation induction, 100 μl of the medium in each well was treated with the final concentrations of the extract of the flower of the Philadelphus schrenckii to 5, 10, and 20 μg/ml, respectively, and further cultured in a cell incubator for 24 hours. After culturing the cells, the experiment was performed using the IL-6 ELISA assay Kit (Rnd System, DY206-05) with the supernatant. The reagent applied in various ELISA assays may be DuoSet Ancillary Reagent Kit2 (RnD system, DY008).

The negative control (NC) group was treated with only serum-free medium without PG-LPS, and the positive control (PC) group was treated with PG-LPS and serum-free medium.

For the medium containing the extract of the flower of the Philadelphus schrenckii, it was confirmed that IL-6, an inflammatory cytokine, was expressed when H₂O₂ was not treated.

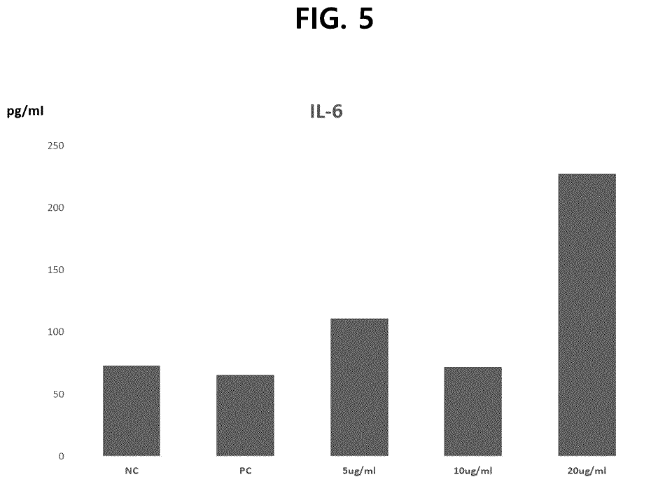

For the negative control, positive control, and 5, 10, and 20 μg/ml of the Philadelphus schenckii flower extract, the amounts of IL-6 expressed are as shown in FIG. 5.

For the medium containing the extract of the flower of the Philadelphus schrenckii, it was confirmed that IL-1B, an inflammatory cytokine, was not expressed when H₂O₂ was not treated.

### Hair on Mammary Papilla Cells Temperament Inhibition Factor Expression Check (BMP2)

To investigate the cytokines of growth inhibitory factors including IL-1β, TGF-β 1, IGF-1, and BMP2, which are representative growth inhibitory factors known to affect the growth inhibition of human dermal papilla cells, and the efficacy of each sample, immunoblot analysis was performed. Human dermal papilla cells (Test Example 1) were seeded at 3×10⁵ cells/well in each well of a 6-well plate containing DMEM medium and cultured in a 37° C. in a 5% CO₂ incubator. After 24 hours, the extract of Philadelphus schrenckii flower was treated to 100 μl of the medium in each well so that the final extract concentrations were 5, 10, and 20 μg/ml, respectively, and cultured for 24 hours. After removing the medium and washing with PBS, the negative control group (NT) was treated with the same volume of distilled water and the positive control group (PC) was treated with 1 uM minoxidil. Cells were lysed with RIPA buffer (Sigma-Aldrich, USA) containing protease inhibitor cocktail (Sigma-Aldrich, USA) and phosphatase inhibitor cocktail, and centrifuged. SDS-PAGE was performed on the same amounts of proteins obtained from the supernatant, and immunoblot analysis was performed using IL-1β antibody (Abcam, USA), anti-TGFβ1 antibody (Abcam, USA), anti-IGF1 antibody (Abcam, USA), anti-BMP-2 antibody (Abcam, USA), anti-DKK-1 (Abcam, USA), and anti-β-actin antibody (Cell Signaling Technology, USA).

Inflammatory response was confirmed by treating H2O2, a type of reactive oxygen species that is an essential mechanism of aging in human hair papilla cells.

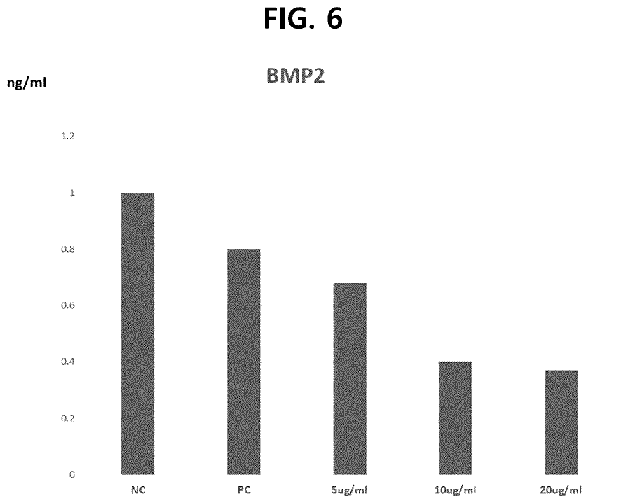

For the negative control, positive control, and 5, 10, and 20 μg/ml of the Philadelphus schrenckii flower extract, the amounts of BMP2 were as shown in FIG. 6.

The BMP2 factor is a biomarker associated with apoptosis, and is a factor known to be produced in the outer root sheath and play a role in promoting the early regression phase of the normal hair follicle growth phase. An increase in BMP2 may indicate cell death. The decrease in the amount of BMP2 expressed as the concentration of the extract of the flower of the Philadelphus schrenckii increases may indicate that apoptosis is inhibited.

What is claimed is:

1. A method for promoting hair growth or preventing hair loss, comprising a step of administering to a subject a composition comprising an effective amount of an extract of Philadelphus schrenckii.

2. The method according to claim 1, wherein the extract is a water or ethanol extract of a flower of the Philadelphus schrenckii.

3. The method according to claim 1, wherein the composition is administered at an amount of 0.1 to 100 mg per day, based on the extract.

4. The method according to claim 1, wherein the composition stimulates a proliferation of dermal papilla cells in hair follicles of the subject.

5. The method according to claim 1, wherein the composition increases the expression of Wnt/β-catenin signaling proteins in the subject.

6. The method according to claim 1, wherein the composition increases the expression of at least one selected from the group consisting of vascular endothelial growth factor (VEGF), Versican, transforming growth factor beta 2 (TGFβ2), hepatocyte growth factor (HGF), glycogen synthase kinase-3 beta (GSK3β), β-catenin, and Wnt5α.

7. The method according to claim 1, wherein the composition is a topical composition.

8. The method according to claim 1, wherein the composition is a cosmetic composition.

9. The method according to claim 1, wherein the composition is a pharmaceutical composition.

10. The method according to claim 1, wherein the composition is a food composition.
